(19) United States
(12) Patent Application Publication
(10) Pub. No.: US 2015/0080999 A1
(43) Pub. Date: Mar. 19, 2015

(54) SELF-EXPANDING STENT

(71) Applicant: ZHEJIANG ZYLOX MEDICAL DEVICE CO., LTD., Hangzhou, Zhejiang (CN)

(72) Inventor: Jonathon Z. Zhao, Hangzhou (CN)

(73) Assignee: ZHEJIANG ZYLOX MEDICAL DEVICE CO., LTD., Hangzhou, Zhejiang (CN)

(21) Appl. No.: 14/383,503

(22) PCT Filed: Oct. 25, 2013
(86) PCT No.: PCT/CN2013/085949

§ 371 Date: Sep. 5, 2014

Related U.S. Application Data
(60) Provisional application No. 61/718,964, filed on Oct. 26, 2012.

(51) Int. Cl. A61F 2/915

(52) U.S. Cl. CPC A61F 2/915; A61F 2002/91558
USPC 623/1.2

(57) ABSTRACT

A self-expanding stent (10), which is created by laser-cutting a Nitinol alloy tubing. A strut pattern of the stent (10) is formed from a continuous helical band that proceeds circumferentially and longitudinally along the length of the stent (10). The helices are formed by repetitions of sinusoidal forms, with a bridge (12) linking the apexes of struts on neighboring adjacent rows directly opposite of each other, for every 4-8 apexes. The linking bridges are substantially straight such that non off-setting pitches is created at the two connected apexes, resulting in a substantially diamond space (71,72,73) between adjacent rows of the struts, instead of a substantially interdigitated space. The strut repetitions are substantially sinusoidal or in a zigzag fashion. The bridges link the apexes of the repetitions forms directly on adjacent rows of struts. The ends of the stent (10) may be formed by using a transition zone on each end that employs gradually decreasing lengths of struts to complete the transition to an even end. The stent (10) made with this pattern and a suitable material has an optimal combination of torsional flexibility, high radial strength and good resistance to longitudinal compression.

### CROSS-REFERENCE OF RELATED APPLICATION

This application claims priority to U.S. application No. 61/718,964, filed on Oct. 26, 2012, the contents of which are incorporated here by reference in their entireties.

### FIELD OF THE INVENTION

The present invention relates to flexible stents that are implanted in a lumen in the body and in particular in blood vessels.

### BACKGROUND OF THE INVENTION

Stents are mesh-like scaffolds which are positioned in diseased and narrowed segments of a vessel to keep it patent or open. Stents are used in angioplasty to repair and reconstruct blood vessels. Placement of a stent in the diseased arterial segment provides structural support to the vessel and prevents elastic recoil and closing of the artery. Stents may be used inside the lumen of any physiological space, such as an artery, vein, bile duct, urinary tract, alimentary tract, tracheobronchial tree, cerebral aqueduct or genitourinary system. Stents may also be placed inside the lumen of human as well as non-human animals.

In general there are two types of stents: self-expanding (SE) and balloon-expandable (BX). Balloon expandable stents are typically made from a solid tube of stainless steel. Thereafter, a series of cuts are made by laser cutting in the wall of a metal tubing. The stent has a first smaller diameter configuration which permits the stent to be delivered through the human vasculature by being crimped onto a balloon catheter. The stent also has a second, expanded diameter configuration, upon the application, by the balloon catheter, from the interior of the tubular shaped member of a radially, outwardly directed force. Inflation of the balloon compresses the arterial plaque and secures the stent in place within the affected vessel. One problem with balloon stents is that the inside diameter of the stent may become smaller over time if the stent lacks sufficient expanding resilience. The result of this lack of resilience is that the stent recoils with the natural elastic recoil of the blood vessel.

In contrast, a self-expanding stent is capable of expanding by itself. There are many different designs of self-expanding stents, including, coil (spiral), circular, cylinder, roll, stepped pipe, high-order coil, cage or mesh. Self-expanding stents act like springs and recover to their expanded or implanted configuration after being compressed. As such, the stent is inserted into a blood vessel in a configuration after being compressed. As such, the stent is inserted into a blood vessel in a compressed state and then released at a site to deploy into an expanded state. One type of self-expanding stent is composed of a plurality of individually resilient and elastic thread elements defining a radially self-expanding helix. This type of stent is known in the art as a “braided stent”. They typically do not have the necessary radial strength to effectively hold open a diseased vessel. In addition, the plurality of wires or fibers used to make such stents could become dangerous if separated from the body of the stent, where it could pierce through the vessel.

Self-expanding stents cut from a tube of super-elastic metal alloy have been manufactured. These stents are crush recoverable and have relatively high radial strength. See, for example, U.S. Pat. No. 6,013,854 to Moriuchi, U.S. Pat. No. 5,913,897 to Corso, U.S. Pat. No. 6,042,597 to Kveen, patent Application WO 01/189421 A2 to Cottone, and U.S. Pat. No. 8,038,707 B2 to Bales. Such self-expanding stents are placed in the vessel by inserting the stent in a compressed state into the affected region, e.g., an area of stenosis. Once the compressive force is removed by pulling back the sheath, the stent expands to fill the lumen of the vessel. The stent may be compressed using a tube that has a smaller outside diameter than the inner diameter of the affected vessel region. When the stent is released from confinement in the tube, the stent expands to resume its original shape and becomes securely fixed inside the vessel against the vessel wall.

Each of the various stent designs that have been used with self-expanding stents has certain functional problems. For example, a stent formed in the shape of a simple circular cylinder does not compress easily. Consequently, insertion of the stent into the affected region of a vessel may be very difficult.

One approach of the prior art stent designs to overcome this problem is to provide a stent formed by zigzag elements as disclosed in U.S. Pat. No. 5,562,697 to Christiansen. A stent formed from a zigzag pattern has flexibility in the axial direction to facilitate delivery of the stent, however, this type of stent often lacks sufficient radial strength to maintain patency of the vessel after elastic recoil.

In order to provide increased radial strength of the zigzag design, the zigzag elements may be connected with connection elements. U.S. Pat. No. 6,042,597 to Kveen et al. describes a balloon expandable stent formed by a continuous helical element having undulating portions which form peaks and troughs where all of the peaks of adjacent undulating portions are connected by curvilinear elements. Connection elements between each adjacent undulating portion may impair flexibility of the stent.

Another approach is to provide a plurality of interconnecting cells which are in the shape of a diamond or rhomboid as in U.S. Pat. No. 6,063,113 to Karteladze et al. or U.S. Pat. No. 6,013,584 to Moriuchi. This type of stent has cells which rigidly interlock. Consequently, these types of stents have a comparatively high degree of rigidity and do not bend to accommodate changes in vessel shape.

Other super-elastic cut-tubular stents has a helically wound configuration of repeating strut patterns. A linking member connects adjacent circumferential windings by extending between loop portions of the sinusoidal forms on adjacent windings. However, the bridge structures and arrangements do not maximize the torsional flexibility of the stents. In particular, WO 01/189421 A2 to Cottone and U.S. Pat. No. 8,038,707 B2 to Bales describe a stent having a helical pattern of bridges (connections) connecting windings of the helix which is reverse in handedness from the undulations of the windings which form the central portion of the stent.

The Cottone design describes a stent having a helical pattern of bridges (connections) connecting windings of the helix which is reverse in handedness from the undulations of the windings which form the central portion of the stent. The design described provides the stent with asymmetric characteristics that cause the stent to resist torsional deformations differently in one direction versus the other. In addition, each “helix of connections” forms a string of connections in which the connections are interrupted by only one and one-half undulations. As such, that string is resistant to stretching and compression. Accordingly, when a stent so designed is twisted torsionally, that string of connections causes constriction of the stent when twisted in the “tightening” direction (i.e., in the direction of the windings) and expansion of the stent when twisted in the opposite “loosening” direction. This differential torsional reaction results in the undulations of the stent being forced out of the cylindrical plane of the surface of the stent, such that the stent appears to buckle when twisted in the “loosening” direction.

In fact, even if the stent were constructed opposite to Cottone's preferred embodiment (that is, with a helix of bridges having the same handedness as the helix of undulations), the same effect results. Stents built with constructions containing a string of bridges separated by only a small number of undulations behave poorly when twisted. That is, they react differently if the stent is twisted one way versus the other, and the surface of the stent tends to buckle when twisted only slightly in the “loosening” direction. Moreover, due to the helical windings of the stents, the stents described by Corso and Kveen terminate unevenly at the end of the helical windings. As such, the terminus of the final winding fails to provide a uniform radial expansion force 360° there around. Cottone addresses this problem by providing a stent constructed with a helically wound portion of undulations in the central portion of the stent, a cylindrical portion of undulations at each end of the stent, and a transition zone of undulations joining each cylindrical portion to the central helically wound portion. The undulations of the transition zone include struts which progressively change in length.

Because the transition zone must mate directly to the cylindrical portion on one side and to a helically wound portion on the other side, the transition zone must create a free end from which the helical portion extends, must contain a bifurcation, and must depart from a uniform strut length for the struts around the circumference of the transition zone so that the transition from the helically wound portion to the cylindrical portion can occur.

However, if there are longer struts in a portion of the transition zone, that portion tends to expand more than the portion with shorter struts because the bending moments created by longer struts are greater than those created by shorter struts. Also, for the same opening angle between two such struts when the stent is in an expanded state, the opening distance between such struts is greater if the struts are longer. These two factors combine their effects in the portion of the transition zone with longer struts so that the apparent opening distances are much larger than in the portion where the struts are shorter. As such, the simple transition zone described by Cottone is not amenable to uniform expansion and compression, which is a requirement of an efficient self-expanding stent.

Moreover, except in the case of the Cottone helical stent which is provided with a transition zone, and except where there are different strut lengths in the undulations at the ends of a stent, stents generally contain struts of one length throughout their design. Accordingly, in order to achieve uniform opening of the stent, all the struts have substantially the same width as well as length.

U.S. Pat. No. 8,038,707 B2 to Bales describes a cut-tube self-expanding stent having a central helically wound portion comprising repeating undulations formed of struts provided at each of its ends with a cylindrical portion, and a transition zone between the helical portion and each cylindrical portion. This patent lists several criteria that provide for better torsional flexibility and expandability in a self-expanding helically wound stent. According to a first criterion, the torsional flexibility of the stent is maximized by having all the “strings” of bridges which connect adjacent helical winding be separated by a maximum number of undulations to make the stent stretchy and compressible. According to a second criterion, the undulations in the central portion are interdigitated to accommodate stent crimping. According to the most preferred embodiment the bridges join loops of undulations which are out of phase by one and one-half undulations. The Bales design suffers a flaw of having the linking bridges being out of phase which will potentially cause the stent to be longitudinally compressed at the deployment site.

There is therefore a great need to further improve the design of a self-expanding stent that overcomes the deficiencies of the prior art stents. An objective of the current invention to provide a geometric design for a stent that has both a high degree of flexibility, significant radial strength and satisfactory resistance to longitudinal compression. The stent is further able to respond dynamically to changes in blood pressure.

### BRIEF DESCRIPTION OF THE DRAWINGS

FIG. 1 is a two-dimensional flattened view of a helical stent according to the invention, wherein the stent is cut parallel to its longitudinal axis and laid flat.

FIG. 2 is an enlarged two-dimensional flattened view of a transition end zone of FIG. 1.

FIG. 3 is an enlarged two-dimensional flattened view of the regular middle portion of the helical stent of FIG. 1.

FIG. 4 is a schematic view of regular middle portion of the helical stent in FIG. 1, showing the direct linking bridges and substantially regularly repeating-diamond shaped space between circumferential windings.

FIG. 5 is a photo of a stent of the current invention, showing the flexibility of a bended stent.

FIG. 6 is a photo of a stent of the current invention, showing the continuous diamond space between the rows of struts.

### SUMMARY OF THE INVENTION

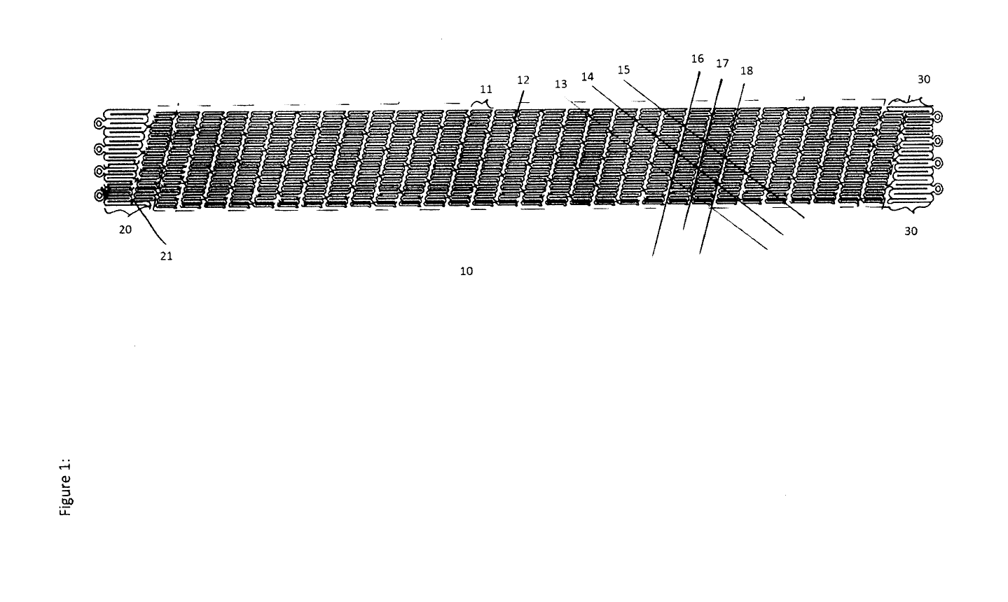

The stent of the invention comprises a self-expanding stent formed from laser cutting a nickel-titanium alloy tube. The stent pattern comprises different types of helices cut from a hollow tube to form the basic supporting structure of a stent. The first helix is formed from a plurality of sinusoidal repetitions (see FIG. 1) and the second type of helix is formed from a plurality of connecting elements such that the bridges connecting the apexes of every few turns of the sinusoidal repetitions. The first and second helices proceed circumferentially in opposite directions along the longitudinal axis of the hollow tube.

The ends of the stent may be formed by a closed circumferential windings of gradually decreasing lengths. The last regular strut is linked by a bridge to the longest strut in the transition end zone while the shortest length strut in the transition zone is linked back to the beginning longest strut. The decreased lengths of the transition zone effect an end plane of the stent that is substantially perpendicular to the longitudinal axis of the stent. The transition zone struts are also linked to the apexes of the struts in the last row of regular middle portion to effect the transition. The width of the transition zone struts is also substantially and gradually larger than those of the middle portion, to compensate the fewer number of struts per surface area on the stent.

Specifically, the invention provides self-expanding stents each including:

a central portion comprised of substantially identical repetitions of helical circumferential windings separated by sufficient helical space, each of the windings including a plurality of sinusoidal waves, with each sinusoidal wave being defined by two adjacent struts and an apex connecting the struts, wherein adjacent windings of the central portion are linked by a plurality of bridges, the bridges extending directly across the helical space between adjacent apexes, wherein the connecting bridges are fewer than all of the sinusoidal waves in the adjacent turns of the circumferential windings, and

a first and second transition end zones connecting the central portion from both ends respectively, the first and second transition zones each including a plurality of transition struts that progressively decrease in length from a longest strut to a shortest strut, and a terminal end of a strut of the central portion adjoining the longest strut to begin the transition,

wherein the stent has a tubular structure having a first smaller diameter for insertion into a vessel, and a second larger diameter for deployment within the vessel.

In some embodiments, the apexes of the sinusoidal waves on adjacent windings are directly linked by bridges. For example, the bridges can extend between apexes are through direct links without off-setting pitches.

In some other embodiments, the central portion of the stent includes fourteen to twenty (e.g., sixteen to nineteen or fourteen to eighteen) sinusoidal waves.

In some other embodiments, each helical winding contains three to five direct bridges extending therebetween.

In some other embodiments, each of the bridges in the central portion extends in a same direction in a cylindrical plane of the stent.

In some other embodiments, the tubular structure is self-expanding from the first diameter to the second diameter.

In some other embodiments, the tubular member is a laser cut tube and made from a super-elastic material.

In some other embodiments, the central portion of the stent comprises of a plurality of helical circumferential windings with struts of a same length and a same width. In some of these embodiments, a direct bridge linking the apexes of the struts in adjacent helical circumferential windings is repeated for every 3-6 struts; direct bridges linking the apexes of the struts in adjacent helical circumferential windings for a helical lines that are crosswise to the helical circumferential windings; the same length of the struts of the central portion is shorter than the shortest strut of the transition end zone, and wherein the same width of the struts of the central portion is narrower than a narrowest strut of the transition zone; the last regular strut of the central portion is linked to the side of the longest strut of the transition end zone; or the bridges linking apexes of struts in the transition zone to the central regular zone is of less frequency than in the middle zone.

In still some other embodiments, each stent further includes a drug-eluting coating on the exterior surface of the stent. Examples of the drug include anti-adhesion compounds, and examples of the coating include biocompatible polymer coatings or macro-organic molecules.

### DETAILED DESCRIPTION OF THE INVENTION

The present invention relates to a self-expanding stent. A stent means any medical device which when inserted into the lumen of a vessel expands the cross-sectional lumen of that vessel. The stent of the invention may be deployed in any artery, vein, duct or other vessel such as a ureter or urethra. The stents may be used to treat narrowing or stenosis of any artery, including, the coronary, infrainguinal, aortoiliac, subclavian, mesenteric or renal arteries.

The term “sinusoidal waves” or “sinusoidal repetition” refer to the bends or undulations in the helical windings forming a continuous helix in the stent. These undulations may be formed in a sinusoidal, zigzag pattern or similar geometric pattern.

The wall may have a substantially uniform thickness. In the compressed state, the stent has a first diameter. This compressed state may be achieved using a mechanical compressive force. The compressed state permits intraluminal delivery of the stent into a vessel lumen. The compressive force may be exerted by means of a sheath in which the compressed stent is placed. In the uncompressed state, the stent has a second variable diameter which it acquires after withdrawal of the compressive force such as that applied by the sheath. Upon withdrawal of the compressive force, the stent immediately expands to provide structural support for the vessel.

The stent is formed from a hollow tube made of super elastic metal. Notches or holes are made in the tube forming the elements of the stent. The notches and holes can be formed in the tube by use of a laser, e.g., a YAG laser, electrical discharge, chemical etching or mechanical cutting. As a result of this type of processing, the stent comprises a single piece that lacks any abrupt change in the physical property of the stent such as that which would result from welding. The formation of the notches and holes to prepare the claimed stent is considered within the knowledge of a person of ordinary skill in the art.

The wall of the stent comprises a scaffolding lattice, where the lattice is formed from two different types of helices. The scaffolding lattice uniformly supports the vessel wall while maintaining deployed flexibility. This design further allows the stent to conform to the shape of the vessel. The first type of helix is formed from a plurality of “sinusoidal repetition” continuously linked together and the second type of helix is formed from a plurality of linking bridges that form a helix that runs crosswise to the first helix formed by the circumferential windings.

The term “bridge” or “linking bridge” refers to the structural element that connects the apexes of struts in adjacent circumferential windings. These bridges are linked together and repeated regularly at a frequency lower than the sinusoidal waves.

These bridges also provide sufficient space between adjacent rows of circumferential windings. The preferred embodiment comprises linking bridges that directly link the apexes in a direct (without off-set or pitches) to optimal space is created therein and longitudinal compressions of the stent is minimized.

FIG. 1 shows a two-dimensional flattened view of the current stent. The central portion of the stent 10 is formed from a first type of helix composed of a plurality of sinusoidal repetition 11. These sinusoidal waves are regularly linked across the adjacent rows by a bridge element 12 with a frequency lower than that of the sinusoidal waves. The gap or space between the adjacent rows of circumferential windings is spaced regularly by the linking bridges 12. The regular patterns of the bridges form a helical pattern (13, 14, 15) that runs crosswise to that of space between the row of stent struts (16, 17, 18).

The stent of the invention also has one transition zone on each end of the stent (20, 30) to make both ends form a plane that is perpendicular to the longitudinal axis of the stent. Such a transitional end zone has struts of gradually decreasing lengths, starting from the longest strut linked to the last strut of the central regular strut (21). These transition struts have decreasing widths that are proportional to the length of the stent to provide radial strength, with the longest strut having the largest width and the last transition strut having the narrowest width. The struts transition zone is linked to the apexes of the last row of regular struts in the middle portion, with a frequency lower than in the middle portion.

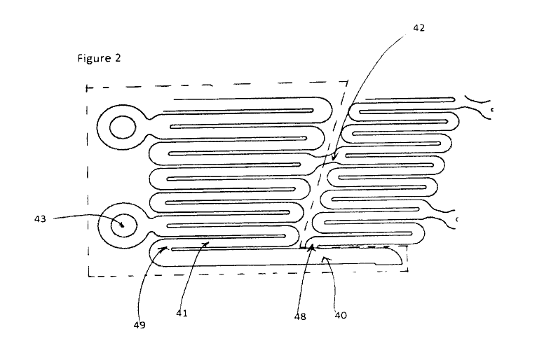

FIG. 2 shows an enlarged two-dimensional flattened view of a transition end zone of FIG. 1. In this figure the transition is defined by the hashed line. The transition strut 40 is linked to the last regular strut in the middle portion of 40 at 48. The linkage of substantially perpendicular such as minimal stress results when the stent is expanded during deployment. The struts in the transition zone form a circumferential winding with gradually decreasing lengths, with the last and shortest strut 41 connected to the longest strut 40 forming the last apex 49. The transitional strut zone is linked to the regular central circumferential windings via links 42, having a higher frequency than in the central portion. The transitional end zone optionally has circular elements 43 attached to the apexes of transition struts, which are optionally filled with radio-opaque materials such as Tantalum or Platinum, or gold, as described in U.S. Pat. No. 6,022,374 to Imran, incorporated herein in its entirety by reference.

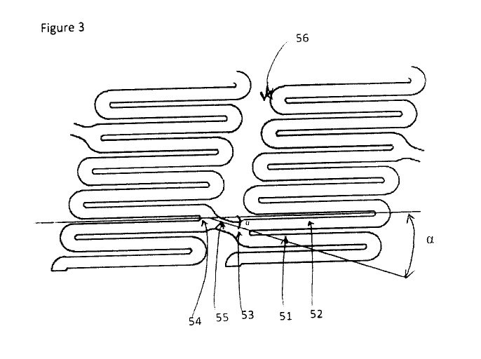

FIG. 3 shows an enlarged two-dimensional flattened view of the regular middle portion of the helical stent of FIG. 1. These repeating circumferential windings have sinusoidal waves of struts having substantially identical struts 51 and 52, linked by turning apex 53 in between. The apexes of adjacent rows of struts 53 and 54 are linked directly via a bridge element 55. The length of the bridge 55 determines the gap or space 56 between two adjacent rows of struts. The longer is the bridge, the bigger is the space. These spaces are of critical importance in that they allow crimping of the stent (compression of the stent diameter) within an outer sheath of a delivery system smoothly without cramping or overlapping of the struts during crimping. The angle alpha of formed by the bridge and the plan of the strut 51 and 52 are preferably low than 45 degree such that the space between the strut will be optimally preserved during deployment and use, providing high resistance to longitudinal compression, which is a potential design drawback of the stent by U.S. Pat. No. 8,038,707 B2 which as an intentionally off-set bridge at about 10 degree pitch. It is believed the stent of this invention will have the optimal combination of flexibility afforded by the repeating circumferential windings, and a sufficiently high resistance to longitudinal compression afforded by these direct linking bridges.

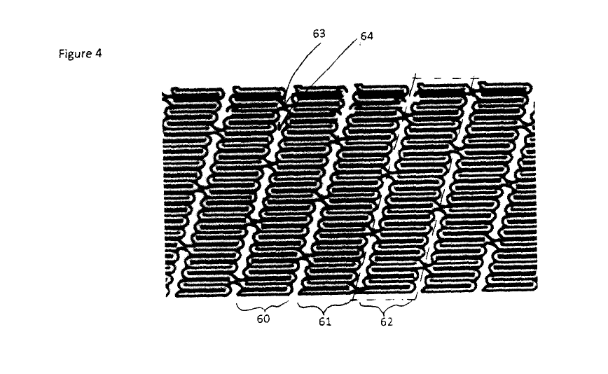

FIG. 4 is a schematic view of regular middle portion of the helical stent in FIG. 1, showing the direct linking bridges 63 and substantially regularly repeating-diamond shaped space 64 between circumferential windings 60, 61, 62. These repeating spaces 64 are created by the direct linking bridges of apexes of adjacent rows of stent struts, and are of repeating diamond shape due to the juxtaposition of the opposing apexes. This feature is in contrast to the prior art stent design such as the one by U.S. Pat. No. 8,038,707 B2 which has linking loops that are offset by a pitch which results in interdigitated loops.

The number of direct connecting bridges connecting two adjacent turns of the helix varies from two to five in each 360 degree turn of the first type of stent helix, depending on the diameter of the stent. In some embodiments, the number of connecting bridges may be greater than four. In all embodiments, the number of connecting bridges connecting adjacent turns of the helix is substantially less than the number of sinusoidal repetitions in one 360 degree turn of the helix.

The length of the repeating struts and the linking bridges in the central portion of the current invention are optimized such as the stent will provide sufficient radial support while retaining a sufficient degree of longitudinal flexibility. In any case, the linking bridges are substantially shorter than the struts.

The scaffolding lattice uniformly supports the vessel wall while maintaining flexibility in a deployed state. This scaffolding lattice confers an anti-crushing property, such that when the stent is crushed radially the stent is capable of rapidly reestablishing its non-crushed state after the crushing force is removed. The scaffolding lattice also allows the stent of the invention to respond dynamically to physiological changes in the blood vessel such as longitudinal shrinkage of the vessel due to elastic recoil or vasconstriction.

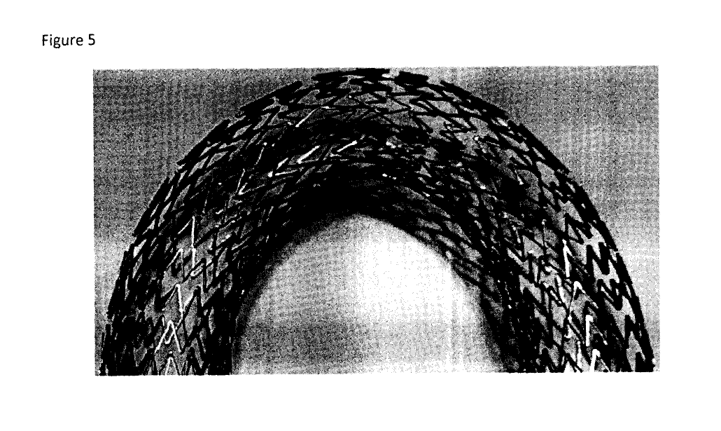

FIG. 5 is a photo of a stent of the current invention, showing the flexibility of a bended stent. With a traditional closed-cell design the bending portion of the stent would have clasped to limit the blood flow through the stent. The current helical design allows the full retention of the patency of the stent while providing adequate surface coverage the bend.

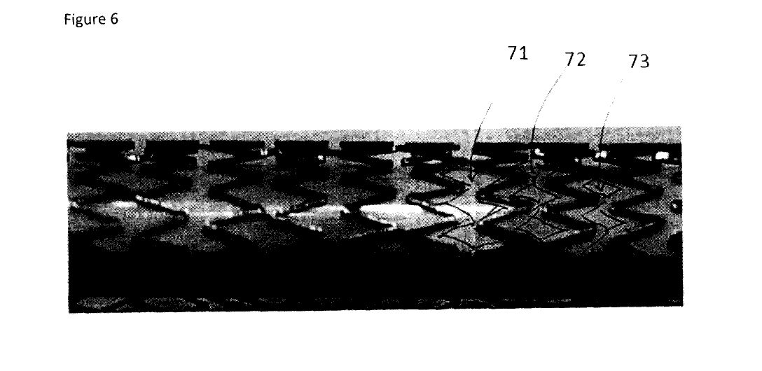

FIG. 6 is a photo of a stent of the current invention, showing the continuous diamond space (71, 72, 73) between the rows of struts. This spacing arrangement provide adequate gap between the rows of the struts and minimized the overlapping of the struts during the crimping and loading processes.

Having described several different embodiments of the invention, it is not intended that the invention is limited to such embodiments and that modifications and variations may be effected by one skilled in the art without departing from the spirit and scope of the invention as defined in the claims.

What is claimed is:

1. A self-expanding stent comprising:
a central portion comprised of substantially identical repetitions of helical circumferential windings separated by sufficient helical space, each of the windings including a plurality of sinusoidal waves, with each sinusoidal wave being defined by two adjacent struts and an apex connecting the struts, wherein adjacent windings of the central portion are linked by a plurality of bridges, the bridges extending directly across the helical space between adjacent apexes, wherein the connecting bridges are fewer than all of the sinusoidal waves in the adjacent turns of the circumferential windings, and
a first and second transition end zones connecting the central portion from both ends respectively, the first and second transition zones each including a plurality of transition struts that progressively decrease in length from a longest strut to a shortest strut, and a terminal end of a strut of the central portion adjoining the longest strut to begin the transition,
wherein the stent has a tubular structure having a first smaller diameter for insertion into a vessel, and a second larger diameter for deployment within the vessel.

2. The stent of claim 1, wherein the apexes of the sinusoidal waves on adjacent windings are directly linked by bridges.

3. The stent of claim 2, wherein the bridges extend between apexes are through direct links without off-setting pitches.

4. The stent of claim 1, wherein the central portion of the stent includes fourteen to twenty sinusoidal waves.

5. The stent of claim 4, wherein the central portion of the stent includes sixteen to nineteen undulations.

6. The stent of claim 1, wherein each helical winding contains three to five direct bridges extending therebetween.

7. The stent of claim 1, wherein each of the bridges in the central portion extends in a same direction in a cylindrical plane of the stent.

8. The stent of claim 1, wherein the tubular structure is self-expanding from the first diameter to the second diameter.

9. The stent of claim 1, wherein the tubular member is a laser cut tube and made from a super-elastic material.

10. The stent of claim 1, wherein the central portion of the stent comprises of a plurality of helical circumferential windings with struts of a same length and a same width.

11. The stent in claim 10, wherein a direct bridge linking the apexes of the struts in adjacent helical circumferential windings is repeated for every 3-6 struts.

12. The stent in claim 10, wherein direct bridges linking the apexes of the struts in adjacent helical circumferential windings for a helical lines that are crosswise to the helical circumferential windings.

13. The stent of claim 10, wherein the same length of the struts of the central portion is shorter than the shortest strut of the transition end zone, and wherein the same width of the struts of the central portion is narrower than a narrowest strut of the transition zone.

14. The stent of claim 10, wherein the last regular strut of the central portion is linked to the side of the longest strut of the transition end zone.

15. The stent of claim 10, wherein the bridges linking apexes of struts in the transition zone to the central regular zone is of less frequency than in the middle zone.

16. The stent of claim 1, further comprising a drug-eluting coating on the exterior surface of the stent.
